(19) United States
(12) Patent Application Publication
(10) Pub. No.: US 2026/0108778 A1
(43) Pub. Date: Apr. 23, 2026

(54) System and Method for Enabling Participation in Random Play Dance Both Online and Offline with Integrated Grading System

(71) Applicants: Youngjae Lee, Chino, CA (US); Joohyung Lee, Corona, CA (US)

(72) Inventors: Youngjae Lee, Chino, CA (US); Joohyung Lee, Corona, CA (US)

(21) Appl. No.: 18/919,104

(22) Filed: Oct. 17, 2024

(51) Int. Cl. A63B 24/00; A63B 71/06

(52) U.S. Cl. CPC A63B 24/0006; A63B 71/06; A63B 2244/22

(57) ABSTRACT

This invention introduces a system for random play dance (RPD) sessions, enabling participation in both offline and online environments with precise performance evaluation and grading. Offline dancers use smartphones equipped with motion and GPS sensors to track accuracy and synchronization, while GPS ensures they remain in the designated area. Online participants rely on AI-powered real-time video analysis to evaluate movements and timing with the music. Optional wearable sensors can further enhance accuracy. The system integrates data from both modes, ensuring consistent evaluation. Augmented reality (AR) enhances the experience by merging online dancers into offline sessions for an immersive RPD experience.

### DESCRIPTION OF INVENTION

The present invention relates to the field of recreational dance activities, specifically to the practice of random play dance (RPD). Random play dance is a popular form of social dancing where participants perform choreographed dance routines to randomly selected songs. Traditionally, participants engage in RPD purely for enjoyment and social interaction, without any formal scoring system, rewards, or economic incentives. The current status of RPD lacks a structured framework for evaluating dance performances, offering rewards, or creating significant economic impact.

Despite its popularity, the absence of an organized grading system or reward mechanism caused that RPD remains primarily a casual and recreational activity. Participants have no method of receiving feedback on their performance, nor is there any incentive structure to motivate improvement or sustained engagement. This limitation constrains the potential for RPD to grow beyond a fun pastime into a more competitive and economically viable activity.

This invention seeks to address these limitations by introducing a comprehensive system that allows participants to join random play dance sessions both online and offline. For offline participants, the system utilizes multiple sensors embedded in cellphones to accurately evaluate and grade dance performances. These sensors can capture a wide range of motion data, enabling precise assessment of each dancer's accuracy and synchronization with the music. For online participants, an AI-based motion detection algorithm is employed to analyze video feeds and grade performances in real-time. And the online participants can join the offline RPD through augmented reality(AR) technology in real-time.

By integrating these advanced technologies, the invention enhances the engagement and enjoyment of RPD, providing participants with valuable feedback and opportunities for improvement. Additionally, the introduction of a structured grading and reward system opens new avenues for economic impact. Dance competitions can be organized with monetary prizes, sponsorships, and other incentives, transforming RPD into a more interactive, competitive, and rewarding activity. This innovation has the potential to significantly elevate the status of random play dance, making it a more structured, appealing, and economically beneficial activity for participants around the world.

### Limitations of Current Existing Methods

Existing dance scoring devices and methods have several limitations that hinder their widespread adoption and effectiveness, particularly for RPD.

Wearable motion tracking system suits, while accurate, present significant challenges. These suits require participants to wear multiple sensors that are restrictive and uncomfortable, detracting from the enjoyment and fluidity of dance movements. Additionally, the high cost of these systems makes them inaccessible for many casual dancers and organizers. The financial burden associated with purchasing, maintaining, and managing these systems limits their use to professional settings or highly-funded events, thus excluding the broader, casual dancing community.

Dancing apps such as Just Dance, although popular, are designed primarily for individual use or small groups in a controlled environment. These apps are not suitable for large gatherings or random play dance scenarios where multiple participants need to be evaluated simultaneously. The current technology used in these apps relies on specific hardware that may not be compatible with all users'devices, creating barriers to participation. Moreover, these apps are designed for entertainment rather than serious performance evaluation, which limits their applicability in competitive or large-scale dance events.

IR motion detection camera systems offer precise motion tracking capabilities, but their practical application is limited due to their prohibitive costs and environmental requirements. These systems necessitate a controlled lab environment to function effectively, with specific lighting conditions and space requirements. The setup and maintenance costs are significant, making these systems impractical for casual or large-scale RPD events. The need for a controlled environment further restricts the flexibility and accessibility of these systems, preventing their use in varied settings such as outdoor events or spontaneous dance gatherings.

Camera-based motion tracking systems, while useful in certain contexts, face challenges when dealing with large crowds, particularly in outdoor or uncontrolled environments. These systems often struggle with accurately tracking multiple participants simultaneously, leading to reduced accuracy and reliability. The complexity of managing and processing data from numerous individuals in a dynamic, uncontrolled environment often results in performance issues. This limitation is especially problematic for RPD events, where numerous dancers perform together in a fluid and unpredictable manner.

The present invention introduces a novel system that enables dancers to participate in random play dance (RPD) sessions from both offline and online environments (100). This system leverages advanced technology to provide accurate performance evaluation, and a structured grading mechanism. The proposed solution addresses the limitations of existing dance scoring devices and methods by integrating cellphone sensors, AI-driven video analysis, and optional external wearable devices.

For offline participants, the system utilizes the motion sensors and GPS sensors embedded in their cellphones to accurately track and evaluate dance performances (101). The motion sensors capture detailed movement data, allowing for precise assessment of the dancer's accuracy, synchronization, and overall performance. The GPS sensor is used to verify the dancer's location and ensure they are within the designated dance area.

To enhance the accuracy and comprehensiveness of the performance evaluation, participants can optionally use external wearable devices equipped with motion sensors and Bluetooth connectivity. These devices, such as smart watches or fitness bands, communicate with the participant's cellphone to provide additional motion data, improving the system's ability to assess complex dance movements and body positions.

For online participants, the system employs an AI-driven real-time video capture and analysis system (102). Participants use their cellphones or other camera-equipped devices to stream their dance performances to the system. The AI algorithms analyze the video feed in real-time, evaluating the participant's movements, synchronization with the music, and overall performance. To overcome the spatial limitations of offline RPD, an online real-time AI motion tracking system can enable participation in offline RPD using AR and digital avatars.

Similar to offline participation, online dancers can optionally use external wearable devices with motion sensors and Bluetooth connectivity. These devices provide supplementary motion data to the AI system, enhancing the accuracy of the performance evaluation. The combination of video analysis and wearable sensor data ensures a comprehensive and precise assessment of the dancer's performance.

The integration component (103) merges data from both offline and online participants, creating a unified platform for tracking and evaluating performances. This system includes a communication interface that synchronizes data from offline motion sensors, GPS, and online video analysis in real-time. Through unified tracking and evaluation algorithms, the system ensures consistent and accurate scoring across both participation modes. Additionally, the integration system facilitates the merging of offline and online dancers in augmented reality (AR), allowing online participants to appear within the offline dance environment, thereby creating a seamless and immersive experience for all participants.

### BRIEF DESCRIPTION OF THE DRAWINGS

FIG. 1. is a schematic diagram of the overall system architecture.

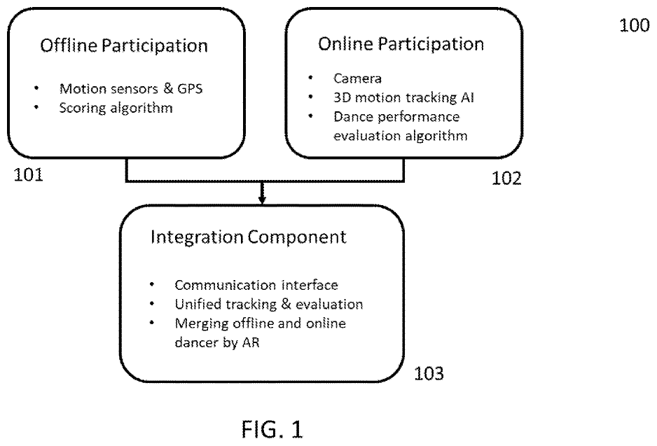

FIG. 2. is a detailed schematic diagram of the offline module.

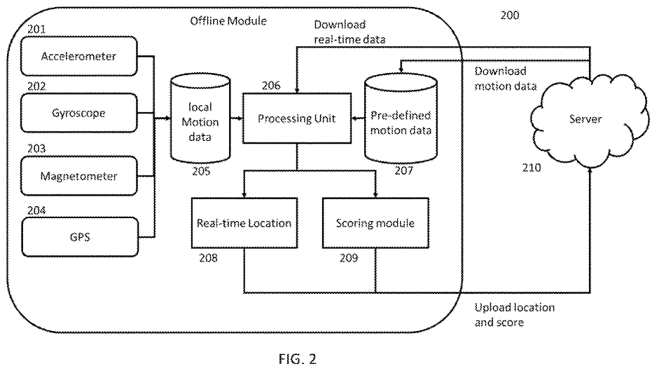

FIG. 3. is a detailed schematic diagram of the online module.

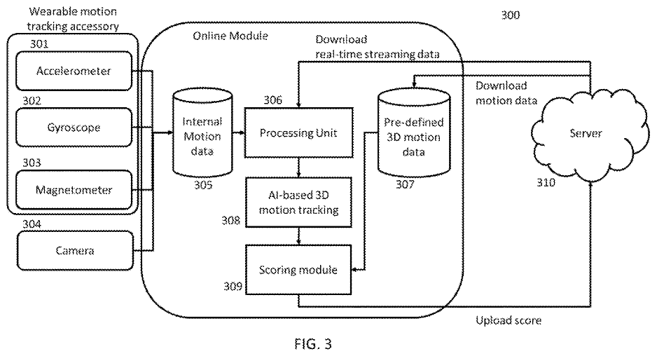

FIG. 4. is a detailed schematic diagram of the integration system.

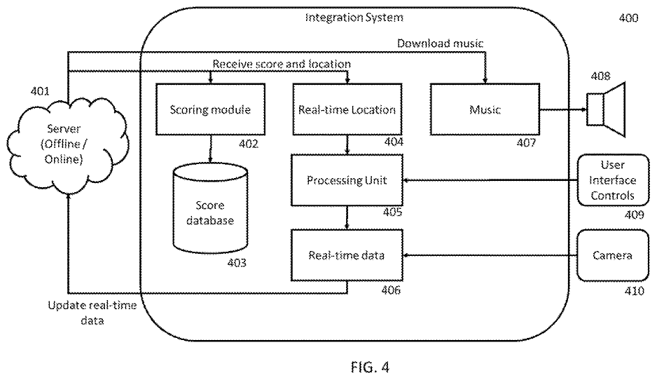

### DETAILED DESCRIPTION

The system for enabling participation in a dance performance evaluation program is designed to function both offline and online, providing a comprehensive and accurate means of performance tracking and evaluation. It consists of three interconnected components: the offline participation module, the online participation module, and the integration component.

The offline participation module (200) is configured to operate in environments where participants can physically join the RPD and perform and record their dance routines in real-time. This module integrates motion sensors, such as accelerometers (201) and gyroscopes (202), magnetometers (203) along with GPS sensors (204), into a mobile device or wearable accessory. These sensors are capable of detecting and recording the participant's movements and location motion data (205) in real-time, capturing detailed information about the dance performance, including speed, rhythm, and spatial orientation. The motion sensors are calibrated to recognize specific dance moves and sequences based on pre-defined motion data (207).

The accelerometer (201) is used to measure the rate of change of velocity of the participant. It is particularly useful in detecting sudden movements, such as changes in speed or direction, which are critical in assessing the intensity and nature of the physical activity.

The gyroscope (202) provides data on the orientation of the participant by measuring the angular velocity. This sensor is essential for understanding rotational movements, such as turning, spinning, or tilting, which are common in various physical activities.

The magnetometer (203) is employed to measure the magnetic field and, when combined with other sensors, helps in determining the participant's orientation relative to the Earth's magnetic field. This is particularly useful for navigation and tracking the direction of movement.

The GPS sensor (204) provides accurate real-time location data (208), enabling the system to track the participant's position during outdoor activities. It can be used to map the participant's route, calculate distance traveled, and assess the overall geographical context of the activity.

Additionally, a scoring module (209) embedded within the offline participation module evaluates the recorded dance performance. This algorithm processes the data captured by the motion and GPS sensors, comparing the participant's movements against predefined criteria such as precision, timing, and execution of dance moves. It operates autonomously, generating a preliminary score for the participant based on real-time analysis.

### Orientation Invariant Algorithm

The AI-integrated scoring module (209) is specifically configured to function effectively regardless of the orientation of the motion sensing device. This capability addresses a significant challenge in activity tracking systems, where the orientation of the device can vary depending on how it is worn, positioned, or used by the participant.

To ensure accuracy across different orientations, the AI-integrated scoring system employs machine learning algorithms and orientation-aware processing techniques. The motion sensing device is equipped with sensors such as accelerometers, gyroscopes, and magnetometers, which continuously monitor the orientation of the device in three-dimensional space. The AI system first detects the current orientation of the device by analyzing the sensor data. Once the orientation is determined, the system normalizes the data by transforming it into a standard reference frame. This normalization process ensures that the data is consistent, regardless of the device's orientation.

The AI-integrated scoring system is trained on a diverse dataset that includes motion data captured in various orientations. During the training process, the AI model learns to interpret the data accurately in any orientation by analyzing examples of the same activity performed with the device in different orientations. The system continuously adapts to new orientations through ongoing learning, refining its understanding of how motion patterns correlate with performance scores, and enhancing the accuracy of the scoring.

Once the data is normalized, the AI system extracts relevant features from the motion data, such as speed, acceleration, rotation, and trajectory. These features are essential for assessing the quality and intensity of the activity being performed. The extracted features are then input into the AI scoring algorithm, which evaluates the performance based on predefined criteria. The AI model generates a score that reflects the participant's performance, considering the activity type and the motion characteristics, independent of the device's orientation.

The system is also capable of providing real-time feedback to the participant based on the AI-generated scores. If the device's orientation changes during the activity, the AI system quickly adapts by recalibrating the scoring mechanism to maintain accuracy. Additionally, the system can alert the participant if the device's orientation significantly deviates from the optimal position, suggesting adjustments to ensure the most accurate tracking and scoring.

The orientation-invariant design of the AI-integrated scoring system offers several key advantages. The system can be used in various activities where the orientation of the motion sensing device may vary, such as in sports, fitness, rehabilitation, or gaming. By normalizing the motion data and using an AI model trained on diverse orientations, the system provides accurate scoring regardless of how the device is worn or positioned. This ensures that participants do not need to worry about maintaining a specific orientation of the device, allowing for more natural and comfortable movement during activities.

### Different Dance of Each Dancer of a Group Dancers

The AI scoring module (209) is also configured to accommodate the complexity and diversity inherent in dance performances. Dance is a dynamic and expressive form of physical activity, often involving intricate sequences of motions that vary greatly between different styles, genres, and individual dancers. In group dance performances, this complexity is further heightened as different dancers may perform distinct motions simultaneously, each contributing to the overall choreography.

To address these challenges, the AI scoring algorithm is designed with several key features that enable it to evaluate multiple dance motions accurately and fairly, even when performed by different dancers within a group.

The AI scoring system begins by capturing motion data from each dancer using the motion sensing devices described in previous claims. These devices, which may include accelerometers, gyroscopes, magnetometers, and GPS sensors, collect detailed information about each dancer's movements, including their speed, orientation, trajectory, and timing. This data is then transmitted to the AI algorithm for processing.

Once the motion data is received, the AI scoring algorithm performs an initial analysis to identify and classify the various dance motions being performed. The algorithm references a comprehensive database of dance movements, which includes a wide range of styles and techniques. This database serves as a benchmark against which the algorithm compares the captured motions, enabling it to accurately identify the specific movements being executed by each dancer.

The algorithm then evaluates each identified dance motion based on predefined criteria, which may include factors such as accuracy, timing, synchronization, fluidity, and expressiveness. For solo performances, the algorithm focuses on the individual dancer's adherence to the expected movements and their overall execution. In group performances, the algorithm takes a more complex approach, evaluating not only each dancer's individual performance but also the coordination and harmony among the dancers.

A key feature of the AI scoring system is its ability to handle scenarios where different dancers perform distinct dance motions simultaneously. In such cases, the algorithm is designed to segregate the motion data for each dancer, allowing it to independently analyze and score the different movements being performed. For example, in a group dance where one dancer performs a series of rapid footwork while another executes fluid arm movements, the algorithm evaluates each dancer's motions separately, referencing the relevant criteria for each type of movement.

Furthermore, the AI scoring algorithm can accommodate variations in choreography where dancers intentionally perform contrasting or complementary motions. In such cases, the algorithm considers the overall artistic intent and the way these distinct motions contribute to the group performance. The system can assess how well the dancers' movements align with the intended choreography, ensuring that the scoring reflects not just technical precision but also the creative and expressive aspects of the dance.

The system also provides feedback to the dancers, highlighting areas where their performance met or exceeded the expected standards and suggesting improvements where necessary. This feedback is tailored to the specific dance motions performed and can be delivered in real-time or as a post-performance analysis, depending on the application.

### GPS Sensor for Location Movement

The offline participation module is equipped with GPS sensors capable of determining the dancer's relative location within a defined performance area. Unlike traditional motion sensors that focus primarily on detecting the dancer's bodily movements (such as acceleration, rotation, and orientation), the GPS sensors specifically track the dancer's position and movement across the physical space. This capability is crucial for evaluating dance performances where spatial dynamics, such as traveling steps, directional changes, and formations, play a significant role in the choreography.

To utilize GPS relative location tracking, the system first establishes a reference framework for the performance area. This framework may be defined by setting GPS coordinates for the boundaries of the space, which could be a stage, rehearsal floor, or any designated dance area. The system calibrates itself to this framework, allowing it to accurately track the dancer's position relative to the established boundaries.

As the dancer moves within this space, the GPS sensors continuously capture location data, which is then transmitted to the offline participation module. The system processes this data in real-time, mapping the dancer's movements and tracking their position changes throughout the performance. The GPS relative location data is then integrated with other motion data, such as acceleration and rotation, to provide a comprehensive picture of the dancer's movement dynamics.

One of the key advantages of using GPS-based location tracking is the ability to incorporate spatial elements into the scoring model. Traditional dance evaluation systems primarily assess movements based on their execution and timing, often neglecting the importance of spatial accuracy and creativity. By including location data, the system can evaluate how well the dancer utilizes the space, adheres to choreography that involves specific spatial patterns, and transitions between different areas of the stage or floor.

The AI-integrated scoring algorithm is specifically designed to leverage this location data in its evaluations. For example, the system can score a dancer's performance based on their ability to accurately execute a sequence of steps that require precise movement across the stage, such as a series of traveling steps or directional changes. The algorithm can also assess how well the dancer maintains their position within group formations, ensuring that they align with other dancers or follow a predetermined path.

In addition to enhancing accuracy, the inclusion of GPS-based location tracking enables the scoring model to account for the complexity of the choreography. For instance, a dance routine that involves intricate spatial patterns, such as diagonal crossings, circles, or zigzag movements, can be more accurately evaluated by analyzing the dancer's adherence to the intended spatial design. The system can detect deviations from the expected path, timing discrepancies in location changes, and overall spatial consistency, all of which contribute to a more detailed and nuanced scoring model.

Furthermore, the system can provide feedback to the dancer based on their spatial performance. If the dancer deviates from the intended path or fails to utilize the space effectively, the system can generate real-time or post-performance feedback, highlighting areas for improvement. This feedback is particularly valuable for dancers and choreographers aiming to refine spatial aspects of their routines.

### GPS Sensor to Distinguish Dancing Area and Waiting Area

The offline dancer's system is specifically designed to manage the spatial organization of dancers within a physical space, such as a dance studio, rehearsal room, or performance venue. This functionality is particularly important in environments where multiple dancers are present, and where it is crucial to maintain order and safety by ensuring that dancers only enter the designated dancing area when it is their turn to perform.

The system incorporates motion sensing devices that are equipped with sensors such as GPS, accelerometers, and magnetometers, as detailed in previous claims. These devices are worn or carried by each dancer and are capable of tracking their precise location within the dance venue. The system continually collects and processes this location data to determine the position of each dancer relative to predefined areas, specifically the waiting area and the dancing area.

To achieve this, the system is configured with a spatial mapping component that defines the boundaries of the waiting area and the dancing area within the venue. This mapping can be accomplished through a combination of GPS coordinates, physical markers, or predefined virtual boundaries stored within the system's database. The boundaries are set up in advance, either by manually inputting the dimensions and coordinates of these areas or by using an automated mapping process.

As dancers move within the venue, the system tracks their locations in real-time. The motion sensing devices continuously transmit location data to the offline participation module, which processes this data to identify whether a dancer is within the waiting area or the dancing area. The system is designed to handle multiple dancers simultaneously, accurately distinguishing between those who are in the waiting area and those who have entered the dancing area.

A critical feature of the system is its ability to provide alerts or alarms when a dancer enters the dancing area. This feature is particularly useful in organized dance settings where dancers must wait for their turn before entering the stage or dance floor. When the system detects that a dancer has moved from the waiting area to the dancing area, it triggers an alarm or alert. This alarm can be in the form of an audible sound, a visual indicator, or a notification sent to a supervisor, instructor, or the dancer themselves.

The alarm system can be customized to suit different scenarios. For instance, in a rehearsal setting, the alarm might serve as a reminder to the dancer that they have entered the active performance space, prompting them to prepare for their routine. In a performance setting, the alarm might notify the stage manager or other staff that a dancer is ready to perform, ensuring smooth transitions between different parts of the performance.

In addition to providing alarms, the system can also generate logs of when dancers enter and exit the dancing area. These logs can be used for various purposes, such as tracking attendance, monitoring rehearsal times, or analyzing the flow of dancers during a performance. The logs can be stored within the system's memory and accessed later for review or analysis.

The system's ability to distinguish between the waiting area and the dancing area and to provide alarms when necessary enhances the overall management of offline dance performances. It ensures that dancers are in the correct location at the appropriate time, reduces the risk of confusion or accidents, and helps maintain the smooth operation of rehearsals and performances.

### Online Participation

The online participation module (300) is designed to facilitate real-time dance performance evaluation using advanced image processing and motion tracking technologies. It incorporates a camera (304) that captures the participant's dance performance in high definition, optimized to function in various lighting conditions and capable of capturing detailed visual data essential for accurate performance analysis. The module also includes a sophisticated AI algorithm that analyzes the visual data captured by the camera in real-time. This AI algorithm utilizes 3D motion tracking technology (308) to accurately map and evaluate the participant's movements, providing an in-depth analysis of the score of dance performance (309). It is trained to recognize and assess complex dance routines, offering real-time feedback and evaluation based on the participant's execution.

### (a) Online Dancers Joining Via Augmented Reality (AR) With Real-Time Streaming

In one embodiment, the system enables online dancers to join offline performances through an augmented reality (AR) interface. This integration allows remote dancers to participate in real-time, as if they were physically present at the venue. The system is designed to provide a seamless and immersive experience by streaming the offline dance performance video directly to the online dancers' AR devices, such as AR glasses, headsets, or mobile devices.

The offline performance is captured using high-definition cameras strategically placed around the venue to provide comprehensive coverage of the dance area. These cameras feed real-time video streams to the system, which then processes and transmits the video to the AR devices of the online dancers. The AR interface overlays the live video feed onto the online dancer's real-world environment, creating the illusion of being present in the offline venue.

Online dancers can interact with the offline dancers in real-time, as the system ensures minimal latency between the live performance and the streamed video. The AR system also includes motion tracking and synchronization features, allowing online dancers to align their movements with those of the offline performers. For example, if the online dancer is performing a routine alongside an offline dancer, the system can track the online dancer's movements and adjust the video feed to ensure that both dancers appear to be moving in harmony.

The AR interface is further enhanced by interactive features such as gesture recognition, voice commands, and virtual controls that allow online dancers to customize their viewing experience, communicate with other participants, or even alter their virtual presence (e.g., changing their avatar or dance style). This integration offers a dynamic and engaging way for online dancers to participate in offline performances, expanding the possibilities for remote collaboration and performance.

### (b) Online Participants Joining Virtually Via Virtual Reality (VR)

In another embodiment, the system allows online participants to join the performance virtually using a fully immersive virtual reality (VR) environment. Unlike the AR setup, where online dancers interact with the real-world performance, the VR experience is exclusively designed for online participants, creating a distinct virtual performance space where they can interact with each other without direct influence from the offline performance.

The VR environment is created using advanced 3D modeling and simulation technologies, replicating or enhancing the offline dance venue in a virtual space. Participants join the VR environment using VR headsets or similar devices. Within this virtual space, online participants can assume the roles of dancers, audience members, or even choreographers, depending on the design of the virtual experience.

The VR system is designed to be highly interactive, allowing participants to move freely within the virtual space, interact with virtual objects, and communicate with other online participants through avatars. The avatars are customizable, enabling participants to choose their appearance, dance style, and even the way they interact with the environment. For instance, a participant may choose to dance alongside others, spectate from different angles, or engage in collaborative choreography sessions.

One of the key features of the VR environment is its ability to simulate various dance scenarios that may not be possible in the offline world. This could include fantastical settings, gravity-defying dance moves, or entirely new dance forms that are enabled by the virtual medium. The system can also host virtual dance competitions, rehearsals, or performances that are attended exclusively by online participants, creating a parallel dance ecosystem that complements the offline world.

The VR environment can also be synchronized with the offline performance, allowing online participants to view a virtual representation of the offline dancers or to participate in a parallel virtual performance that mirrors the offline choreography. This dual-mode capability ensures that the VR experience is both independent and connected to the offline performance, providing a versatile platform for virtual dance activities.

### (c) Integration and Synchronization

The system's AR and VR components are designed to work in harmony, ensuring that both online and offline participants can engage in a unified dance experience. The system includes synchronization algorithms that align the timing, movements, and interactions of participants across both platforms, minimizing latency and ensuring a cohesive experience.

In conclusion, the system offers an approach to integrating online and offline dance performances. Through the use of AR and VR technologies, the system provides flexible and immersive options for online dancers and participants, enabling them to engage with offline performances in real-time or within a fully virtual environment. This innovation opens up new possibilities for remote collaboration, participation, and creativity in the world of dance, ensuring that distance is no longer a barrier to artistic expression.

### (d) Wearable Motion Tracking Accessories

The online participation module might integrate wearable motion-tracking accessories (301, 302, 303) that significantly enhance the accuracy of the scoring algorithm used within the system. These wearable devices, which may include sensors embedded in wristbands, gloves, or other body-worn accessories, capture real-time motion data from users. By accurately tracking the user's movements, these devices enable the system to analyze physical activity with a high degree of precision. The collected motion data is then processed by the module's algorithms to assess user performance, ensuring that the scoring is based on detailed and objective criteria. This integration of wearable technology not only improves the fidelity of the scoring mechanism but also allows for a more immersive and responsive user experience.

Additionally, the online participation module may feature a motion-driven user interface (UI) that leverages the captured motion data for interaction purposes. Users can navigate, select options, and interact with the system's features using natural gestures or movements, providing a hands-free, intuitive control method. This motion-driven UI enhances user engagement, making the module more accessible and user-friendly, especially in environments where traditional input methods may be inconvenient. The combination of wearable motion-tracking technology with a motion-driven UI creates a seamless and interactive experience, optimizing both the functionality and usability of the online participation module.

### Integration System

The integration system (400) is engineered to include a sophisticated communication interface that synchronizes data from both offline and online participation modules (401) into a centralized system. This central system acts as the core of the entire operation, allowing for unified performance tracking and evaluation of all participants. By gathering data from both offline and online dancers, the system is able to store and compare scores (402) in a cohesive manner. This unified approach ensures that all participants, regardless of whether they are engaging online or in person, are evaluated on the same criteria, thereby promoting fairness and consistency in the scoring process.

The integration system is also equipped with the capability to download music (407) from a server and play a playlist across both online and offline environments. This ensures that all participants, whether in the physical venue or participating remotely, experience the same music. By managing music delivery centrally, the system reduces potential delays or discrepancies in music playback, contributing to a more cohesive and fair competition environment.

Furthermore, the integration system is equipped with an advanced camera system (410) designed to enhance the experience of online dancers through augmented reality (AR). This system streams live video of offline dancers, allowing online participants to interact and engage with the competition in a more immersive manner. The AR experience creates a bridge between the physical and virtual environments, enabling online dancers to feel as though they are part of the live event. This integration of AR technology not only enriches the user experience for online participants but also fosters a more inclusive and connected competition environment.

Additionally, the integration system empowers the host to maintain full control over the dance competition through a set of user interface controls (409). The host can make adjustments to the competition, such as modifying rules, initiating or halting rounds, and setting evaluation criteria, all of which are instantly reflected in the system. These real-time updates are communicated to both offline and online dancers, ensuring that all participants are immediately aware of any changes. This capability for real-time host control and participant notification is essential for maintaining the smooth operation of the competition, ensuring that all participants are synchronized and informed, regardless of their mode of participation.

What is claimed

1. A system for enabling participation in a dance performance evaluation program, comprising:
(a) an offline participation module comprising: (i) motion sensors and GPS sensors integrated with a mobile device or wearable accessory for detecting and recording the movements and location of participants in real-time; (ii) a scoring algorithm configured to evaluate the recorded dance performance based on predefined criteria;
(b) an online participation module comprising: (i) a camera for capturing the participant's dance performance; (ii) a real-time 3D motion tracking AI algorithm for analyzing the captured performance;
(c) an integration system comprising: (i) a communication interface to synchronize data from both the offline and online participation modules to a central system for unified performance tracking and evaluation.

2. The system of claim 1, wherein the motion sensors in the offline participation module comprise a combination of one or more motion sensors selected from the group consisting of an accelerometer, a gyroscope, a magnetometer, and a GPS sensor.

3. The system of claim 1, wherein the AI integrated scoring system is configured to be applicable for different orientations of the motion sensing device.

4. The system of claim 1, wherein the AI scoring algorithm is configured to evaluate dance performances by referencing multiple dance motions, accommodating group dances where different dancers may perform distinct dance motions.

5. The system of claim 1, wherein the offline participation module uses GPS relative location to detect the dancer's location movement while dancing, enabling a more detailed scoring model that accounts for location changes during the performance.

6. The system of claim 1, wherein the offline dancer's system is further configured to collect the locations of all offline dancers and distinguish between a waiting area and a dancing area, and to provide an alarm when a dancer enters the dancing area.

7. The system of claim 1, wherein:
(a) online dancers can join via augmented reality (AR) with real-time streaming of the offline dance performance video; or
(b) online dancers can join virtually via virtual reality (VR) exclusively with other online participants.

8. The system of claim 1, wherein the online dancers might use wearable motion tracking accessory.
